(19) United States
(12) Patent Application Publication
(10) Pub. No.: US 2026/0024640 A1
(43) Pub. Date: Jan. 22, 2026

(54) SYSTEM AND METHOD IMPLEMENTED BY COMPUTER FOR THE CONTROL OF ANESTHETIC FLUIDS THROUGH FUZZY CONTROL

(71) Applicant: SmartTIVA Inc, Rockville, MD (US)

(72) Inventor: Juan Manuel FERNANDEZ MONCADA, Rockville, MD (US)

(73) Assignee: SmartTIVA Inc, Rockville, MD (US)

(21) Appl. No.: 18/875,372

(22) PCT Filed: Apr. 26, 2023
(86) PCT No.: PCT/IB2023/054264

§ 371 Date: Dec. 16, 2024

(30) Foreign Application Priority Data
May 29, 2022 (CO) NC2022/0007433

(51) Int. Cl. G16H 20/17; G05B 13/02; G16H 40/63

(52) U.S. Cl. CPC G16H 20/17; G05B 13/0275; G16H 40/63

(57) ABSTRACT

The present disclosure describes embodiments of a computer-implemented method for controlling an anesthetic fluid flow executed on a computer unit and a system, computer-readable media, and computer program associated with the execution of said method. In the method, an input data packet including demographic data and physiological data of a user, and target concentration data is received. Further, the method obtains a first value of a configuration data with a pharmacokinetic processing method from the input data packet. Also, the first configuration data in the method is entered into an anesthetic fluid infusion pump, and a status data is received from an instrumentation system. The status data includes records of at least one variable associated with the anesthetic status and brain condition of the patient. Additionally, the method takes into account a threshold range that is obtained from a pharmacodynamic processing method. Furthermore, the method has a step of obtaining a second setting data value by performing a control process that selectively takes as input the first setting data value, the state data, and the threshold, and performs a PID control method and/or a fuzzy control method to get the second value of the configuration data. Then, the configuration data with the second value is sent to the anesthetic fluid infusion pump to modify its flow conditions. The method iterates the stages in which the setting data value is recalculated based on the state of the user under anesthesia.

### RELATED TECHNICAL FIELD

The present invention relates to computer-implemented systems and methods for fluid flow control. In particular, it relates to computer-implemented systems and methods for the control of anesthetic fluids.

### DESCRIPTION OF THE PRIOR ART

Document CO 15237754 discloses a closed-loop intravenous total anesthesia delivery system and method. This document mentions a step of collecting the information of vital signs and anesthetic depth of a patient, and a step of processing the collected information. Said processing includes choosing the medicine to be used, calculating the medicine peak time, and acquiring the signal of the effect induced by said medicine in terms of anesthetic depth, determining whether the anesthetic depth of the patient is general anesthesia or sedation, and adjusting the medication according to the evolution of the patient. Said document further mentions a step of collecting the heart rate and mean arterial pressure values of the patient and a step of determining the increase or decrease of the medicine effective site concentration of the patient in maintenance phase using a fuzzy logic model. Said document indicates that the anesthetic depth state of the patient is compared against a control state determined by fuzzy logic and determines said increase or decrease of the medicine concentration values. The information on the values of heart rate and mean arterial pressure is entered into said fuzzy logic model, which are interpreted as the level of pain in the patient.

Document U.S. Pat. No. 8,038,642 mentions a system for loop-assisted anesthesia administration that uses electroencephalographic monitoring for dosage adjustment and displays it through a planning system, similar to a clinical decision support system (CDSS). However, this document mentions anesthesia with inhaled gases as the central axis of anesthetic management and indicates that the EEG signal is used to adjust the concentration of said gases.

On the other hand, document U.S. Pat. No. 10,130,766 mentions a system and method that provides closed-loop sedation, anesthesia or analgesia by monitoring the EEG and automatically adjusting the administration of sedative, anesthetic and/or analgesic drugs to maintain the desired level or cortical default at all levels of attention. The control based on EEG, only takes into account one index, and the method only uses this index to reduce or turn off the medicine infusion based on the presence of said index. Moreover, the disclosed system comprises a closed-loop system that does not contemplate the intervention of an expert user for the approval of said adjustments, increasing the risks of overdosage.

On the other hand, the document U.S. Pat. No. 9,849,241 B2 discloses a control method for the administration of medicines for anesthesia that has a step of acceptance by the user based on the interpretation of EEG automatic control signals (closed loop) and semi-automatic control (open loop). In this patent, the EEG measurement only takes into account the index and this index is directly correlated with a concentration in the brain to adjust the model. As the figures show in this document, the analysis of the EEG monitor-derived variable is never directly entered or plugged into the control model, but rather is used as a fitting covariate within the pharmacokinetic model. Also, this document mentions IMS indicator measurement, if you plug in some of the diagrams and fit the model in real time. However, IMS is an exhaled measurement of blood-injected medicine concentration, so it represents a pharmacokinetic, but non-pharmacodynamic, adjustment.

On the other hand, the document Tian, Ye & Chu, Zheng & Ma, Gang. (2022). Fuzzy logic control theory in clinical anesthesia. Expert Systems. 39. 10.1111/exsy.12761 discloses a control method based on the application of fuzzy logic control theory in clinical anesthesia that allows for reducing the risk of clinical anesthesia, e.g., risk of hypotension. Said document mentions a mathematical model of the clinical anesthesia control system. Model parameters are adjusted using a time domain analysis method to measure dynamic features. On the other hand, the system stability is analyzed using the root locus method.

Said document states that predictive fuzzy composite intelligent control is applied to atmospheric and vacuum equipment and mentions a simulation module based on simple loop and cascade control to verify that predictive fuzzy control can improve atmospheric and vacuum equipment. Said document indicates that the control problem is hysteresis and non-linearity. Said document mentions the use of the bispectral index (BIS) as a parameter for monitoring anesthesia depth.

Additionally, said document discloses a closed-loop anesthesia control system that performs feedback control of the anesthesia effect. The closed-loop anesthesia control system provides feedback on the effect of the patient after the execution of the instruction above to the infusion pump, which regulates the infusion rate of anesthetics through a computer, compares it with the preset amount, and guides the output depending on the error between them. Said document discloses that the operating point of the anesthesia system is approached by the preset amount to adjust the drug delivery and anesthesia depth method. Said document mentions that the main difference between the closed-loop anesthesia control system and the traditional anesthesia control system is based on whether the infusion pump accepts the feedback information of the previous execution order.

Said document also mentions that anesthesia periodic monitoring is carried out to determine heart rate (HR), non-invasive blood pressure (NIBP), electrocardiogram (ECG), oxygen saturation at the fingertip (SpO2) and directional recovery after surgery.

Said document also discloses that in cascaded and single-loop analog control the response speed of predictive fuzzy control is faster than that of PID control and fuzzy control, and the stability time is shorter. The predictive fuzzy PID controller can effectively solve the inertia and delay caused by atmosphere temperature control and vacuum devices, and allows for improving the dynamic features, static features, and robustness of its control system.

On the other hand, document US 2019/0374158 A1 discloses a method for monitoring a patient undergoing an administration of at least one drug with anesthetic properties using a monitoring system. The method comprises a step of receiving data corresponding to EEG signals acquired from a patient and an indication of at least one feature of the patient and of the at least one drug with anesthetic properties. Furthermore, the method has a step of assembling one or more sets of EEG time series using the data received, and a step of selecting the alpha frequency signals from one or more sets of EEG time series. The method also has a step of analyzing the alpha frequency signals to determine the particular signatures of the at least one administered drug, based on the signatures and the indication, and a step of identifying at least one of the current states and an expected future state of the patient induced by the at least one drug. Likewise, said document mentions a step of generating a report indicating at least one of the current states and the expected future state of the patient.

Said document further mentions a processor configured to review the physiological data of the plurality of sensors and the indication of the user interface. The processor is also configured to assemble the physiological data into time series data sets and analyze the time series data sets to determine signature profiles consistent with the administration of at least one drug with anesthetic properties, wherein the signature profiles are determined using signals from the time series data in an alpha frequency range.

Additionally, said document mentions that the method can perform a pre-processing algorithm that analyzes indicators, e.g., burst and suppression intervals in the EEG waveform to convert them into a binary time series. This time series is preferably a “real-time” series that is provided as input to a brain state estimation algorithm. One such brain state estimation algorithm is the BSP algorithm, which provides a second-by-second estimate of the burst suppression state of the brain using the concept of a state space model for observing binary and point processes.

Said document discloses a closed-loop drug delivery and control system that performs the BSP algorithm to obtain automatic control of medical coma by closed-loop regulation of an anesthetic drug to maintain a specified level of medical coma in terms of a specified level of BSP burst suppression. Said document also mentions that said BSP algorithm considers a pharmacokinetic model and discloses that a dynamic estimate of the BSP can be used as negative feedback to generate the error signal that enters a proportional-integral (PI) controller.

On the other hand, document U.S. Pat. No. 7,925,338 B2 discloses a method implemented by computer to determine the anesthetic state of a patient. The method comprises a step of establishing a first index value that indicates the current anesthetic state within a first predetermined range of values for a first diagnostic index that indicates the hypnotic level in the patient, and establishes a second index value for the current anesthetic state within a second predetermined range of values for a second diagnostic index that indicates the analgesia level in the patient. The method of said document further has a step of using the first and second index values to indicate the anesthetic state of the patient, wherein the first and second index values are indicated to the user through an indicator unit displaying at least two trend curves, each trend curve representing the values of a corresponding diagnostic index during a preceding period of certain duration.

Said document mentions a control unit that controls a drug delivery system comprising a first supply unit for supplying hypnotic drugs and a second supply unit for supplying analgesic drugs. Said document discloses that the anesthetist can operate the system through a user interface comprising a user input device from which the anesthetist can supply input parameters to the control unit. Furthermore, said document indicates that the supply system may comprise a single supply unit. Therefore, it is also possible that the state of the patient in the two-dimensional state is used to control the administration of an analgesic drug or a hypnotic drug only.

Said document also discloses that measured physiological data of a patient is supplied to a patient condition determination unit comprising, at least at one logic level, a hypnosis index determination unit, and a unit for determining the analgesia index. The former determines the hypnosis index and supplies it to the control unit, while the latter determines the nociception index and supplies it to the control unit.

The control unit determines the location corresponding to the condition of a patient on a two-dimensional diagram and displays the location on the screen of the display unit. The control unit retrieves drug delivery data, such as amounts delivered from the drug delivery system and stores the data to set new parameters for the delivery unit.

Said document mentions that the control unit may also have an independent database containing information on the pharmacodynamic and pharmacokinetic properties of the drugs to be administered. Furthermore, said document discloses that, in the case of an intravenously administered drug, the delivery unit usually includes a motor-driven infusion pump and mentions that the control unit may be a PID controller.

### BRIEF DESCRIPTION OF THE INVENTION

The present invention describes embodiments of a computer-implemented method and system for controlling a flow of anesthetic fluid. The method comprises performing in a computing unit a step a) of receiving an input data packet including demographic data and physiological data of a user, and target concentration data of an anesthetic substance, wherein the input data packet is entered by an operator through an input/output device connected to the computing unit. Furthermore, the method has a step b) of obtaining a first configuration data value by performing a data processing method. Said data processing method is configured to perform a pharmacokinetic processing method taking as input the input data packet and a pharmacokinetic database.

Also, the method has a step c) of inputting the first configuration data into an anesthetic fluid infusion pump, wherein the first configuration data sets an anesthetic fluid flow operating condition, and a step d) of receiving a state data from an instrumentation system. The state data may include records of at least one variable selected from the group including, state variables of the user under anesthesia, electroencephalographic signals, electromyography signals, data derived from electroencephalographic and/or electromyography signals, and combinations thereof.

Additionally, the method has a step e) of obtaining an activation data by means of a validation process that takes as input the state data and a predetermined threshold value related to a user anesthetic state, wherein the activation data is generated when at least one of the state data variables has a value outside the predetermined threshold range. The predetermined threshold range is obtained by performing a pharmacodynamic processing method that takes as input the input data packet and a pharmacodynamic database. Likewise, the predetermined threshold range (13) is obtained from a pharmacodynamic database that includes values of variables reported in the literature, for different levels of anesthetic depth of a population of patients.

Furthermore, the method has a step f) of obtaining a second configuration data value by performing a control process that selectively takes as input the first configuration data value, the state data and the predetermined threshold, and performs a method of PID (Proportional-Integral-Derivative) control that takes as input the first value of the configuration data, the state data and the predetermined threshold, and/or a fuzzy control method, which can take as input an output data of the PID control method. Said control process allows for having the second value. Also, the method includes a step g) of sending the configuration data with a second value to the anesthetic fluid infusion pump to modify the anesthetic fluid flow conditions and, finally, the method iteratively repeats steps c) to g), e.g., during the time determined for a medical procedure undergone by the user (patient).

On the other hand, the system disclosed in this document includes at least the computing unit configured to perform any of the methods described herein. Optionally, the system may further include one or more elements selected from the input/output device, the anesthetic fluid infusion pump, and the instrumentation system.

In addition, the present invention describes a computer-readable media that includes instructions that, when executed by a computing unit, causes said computing unit to perform any of the embodiments of the method disclosed herein.

Likewise, this invention describes a computer program that includes instructions that, when executed by a computing unit, causes said computing unit to perform any of the embodiments of the method disclosed herein.

The methods, systems, computer-readable means, and computer programs disclosed herein allow, when any of the embodiments of the method described herein is executed to efficiently control the intravenous administration of a substance that induces a user (patient) to an anesthetic state. In particular, the method disclosed herein takes into account variables that reflect a more complete state of brain function, and not only their static value and scale are compared, but also the trend that these values have had in each user and in each procedure to which users are subjected. With these trends and values, a pharmacodynamic model can be applied that interprets and calculates by means of fuzzy logic the value of the configuration data that modifies the infusion rate of the anesthetic fluid when received by the anesthetic fluid infusion pump.

### BRIEF DESCRIPTION OF THE FIGURES

FIG. 1 shows a block diagram of one embodiment of the method and system disclosed herein, in which a computing unit connected to an input/output device, an anesthetic fluid infusion pump, are identified and details of a data processing method and a control process that modify the values of the configuration data affecting the flow of anesthetic fluid of said anesthetic fluid infusion pump are shown.

FIG. 2 shows a flowchart of an embodiment of the method disclosed herein in which a conditional is identified in which the operator intervenes to accept the sending of the configuration data value to the anesthetic fluid infusion pump.

FIG. 3 shows a flow diagram of an embodiment of the method disclosed herein, in which it is shown that by displaying the value of the configuration data in the input/output device, the operator manually enters said value into the anesthetic fluid infusion pump.

FIG. 4 shows an example of a screen in which the setting data value is displayed.

### DETAILED DESCRIPTION

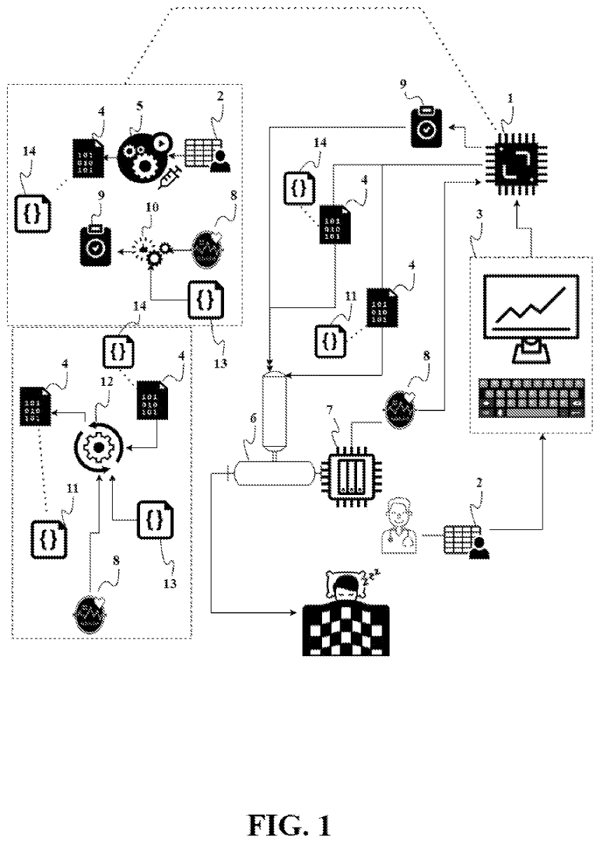

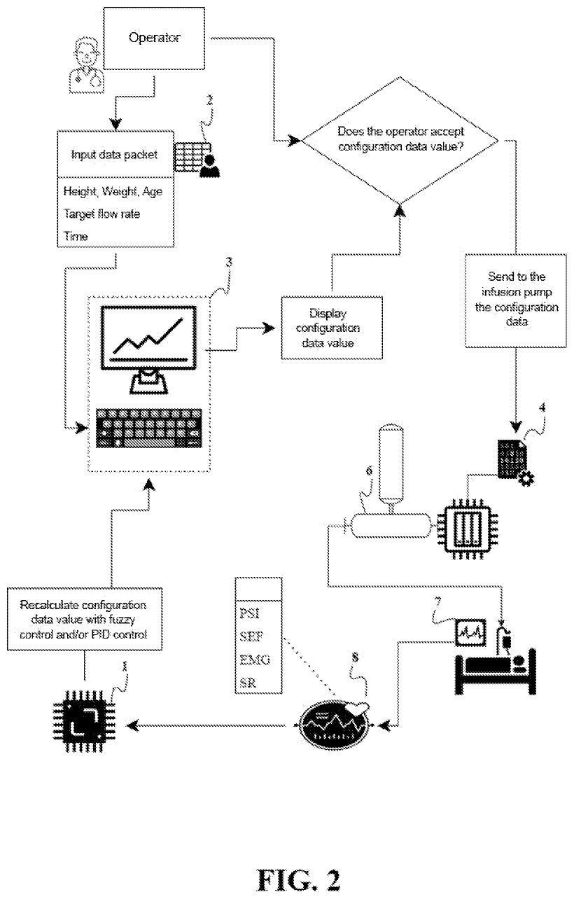

Referring to FIG. 1 and FIG. 2, the present invention describes embodiments of a computer-implemented method and system for controlling a flow of anesthetic fluid.

The method comprises performing in a computing unit a step a) of receiving an input data packet including demographic data and physiological data of a user, and target concentration data of an anesthetic substance, wherein the input data packet is entered by an operator through an input/output device connected to the computing unit. In addition, the method has a step b) of obtaining a first value (14) of a configuration data (4) by performing a data processing method (5) configured to perform a pharmacokinetic processing method that takes as input the input data packet (2) and a pharmacokinetic database.

The pharmacokinetic database can be represented within a pharmacokinetic model (e.g., a set of one or more mathematical functions that correlate variables associated with the pharmacokinetics of an anesthetic substance or medicine in the body of a population of users) obtained with methods of statistical and mathematical processing taking as input the pharmacokinetic database. In addition, the pharmacokinetic database can be a data structure that includes variables associated with the pharmacokinetics of an anesthetic substance or medicine in the body of a population of users (patients).

Also, the method has a step c) of entering the first configuration data (4) in an anesthetic fluid infusion pump (6), wherein the first configuration data (4) sets anesthetic fluid flow operating conditions, and a step d) of receiving state data (8) from an instrumentation system (7). The state data (8) may include records of at least one variable selected from the group that includes, state variables of the user under anesthesia, electroencephalographic signals, electromyography signals, data derived from electroencephalographic, and/or electromyography signals, and combinations thereof.

Examples of variables contained in the state data (8) are PSI (patient state index), SEF (spectral edge frequency), EMG (electromyography), bi-spectral index (BIS), and SR (suppression rates), and combinations thereof. In particular, the values of PSI, SEF, SR, and BIS are indicators of the anesthetic state of the user (patient).

Referring to FIG. 1, additionally, the method has a step e) of obtaining activation data (9) through a validation process (10) that takes as input the state data (8) and a predetermined threshold range (13) related to an anesthetic state of the user, where the activation data (9) is generated when at least one of the variables of the state data (8) has a value outside the predetermined threshold range (13). The predetermined threshold range (13) is obtained from a pharmacodynamic database:

In addition, the method has a step f) of obtaining a second value (11) of the configuration data (4) by performing a control process (12) that selectively takes the first value (14) of the configuration data (4), the state data (8), and the predetermined threshold (13), and performs a PID (Proportional-Integral-Derivative) control method that takes as input the first value (14) of the configuration data (4), the state data (8) and the predetermined threshold (13), and/or a fuzzy control method, which can be taken as taking an output data of the PID control method as input. Said control process (12) allows for having the second value (11). Also, the method includes a step g) of sending the configuration data (4) with a second value (11) to the anesthetic fluid infusion pump (6) to modify the anesthetic fluid flow conditions, and finally, the method iteratively repeats steps c) to g).

It will be understood in the present invention that the first value (14) and the second value (11) are values of the configuration data (4) in different iterations of the method. In particular, the first value (14) could be understood as a value “f(i)” and the second value (11) as a value “f(i+1)” where “i” is the current iteration of the method. According to the foregoing, when the second value (11) is obtained and it is returned to step c), said second value (11) becomes the first value (14) of the next iteration.

In particular, the method disclosed herein takes into account variables that reflect a more complete state of brain function, and not only its static value and its scale are compared, but also the trend that these values have had in each user and each procedure to which users are subjected. With these trends and values, a pharmacodynamic model is applied that interprets and calculates, by means of fuzzy logic, configuration data (4) value (11, 14) that modifies the infusion rate of the anesthetic fluid when received by the anesthetic fluid infusion pump (6).

As set forth above, the method disclosed herein can be understood as a clinical decision support computer-implemented method (CDSS) for real-time control of total intravenous anesthesia (TIVA), based on a fuzzy logic pharmacodynamic model.

In the present invention, data will be understood as a symbolic representation that can be numerical, alphabetic, algorithmic, logical, and/or vector that encodes information. A piece of data can have a structure or frame comprising blocks of characters or bits that represent different types of information. Each block comprises strings of characters, numbers, logical symbols, among others. A piece of data can also comprise only bits (strings in binary language), comprise characters formed one by one by a combination of bits, comprise fields, records or tables formed by fields and records, or formed by data interchange files (formats such as csv, json, xls, among others). Furthermore, a data item can be a matrix of n rows by m columns. In turn, a data can contain several data. For example, when the data has a frame structure, the frame may have a block of identifying characters, generally known as a header, which contains information related to a computing device or processor that sends the data and may contain information related to a computing device or processor that receives the data. Preferably, if data is in a frame format, the frame contains blocks related to layers according to the OSI reference model.

Likewise, the frame can have a block of tail characters (or simply tail), which allows for the identification of a computing unit or server that is the end of the data, i.e., after that block the information contained in the data previously identified by the computing unit or server with the header is no longer found. In addition, the data has one or more blocks of characters between the header block and the tail block that represent statistics, numbers, descriptors, words, letters, logical values (e.g., Booleans) and combinations thereof.

On the other hand, the operator can be a health professional who monitors the user (patient) during a medical procedure in which the user is in an anesthetic state. For example, the operator can be an anesthesiologist. The operator inputs into the input/output device (3) the input data packet (2) with user variables that make it possible to determine the dose of anesthetic drug that must be delivered by the anesthetic fluid infusion pump (6). Said variables can be, e.g., weight, sex, age, height, clinical history data (allergies to medicines, other medicines consumed by the user, history of pathologies, among others) and other physiological, medical and/or demographic data of a user who is sought to be kept under sedation (patient). The method allows for the operator to be assisted by suggesting values of the configuration data (4) automatically, which affect the anesthetic state of the user (patient).

For example, in any of the embodiments of the method disclosed herein, the target concentration data may be a concentration value of an anesthetic substance selected from the group that includes hypnotic, analgesic and amnestic intravenous anesthetic drugs, paralytic agents, vasodepressors, pressor substances, and formulations with combinations thereof. Examples of substances included in the anesthetic fluid that can be administered by the anesthetic fluid infusion pump (6) can be Propofol, Fentanyl, Remifentanil, analgesics (e.g., opioids), antibiotics, muscle relaxants (e.g., Rocuronium, Artracuronium) and formulations with combinations thereof.

On the other hand, the method has a step b) of obtaining a first value (14) of a configuration data (4) by performing a data processing method (5) configured to perform a pharmacokinetic processing method that takes as input the input data packet (2) and a pharmacokinetic database.

The pharmacokinetic processing method is a computational process executed by the computing unit (1) that takes as reference a pharmacokinetic model and the variables associated with the user under anesthesia that are in the input data packet (2).

In both the pharmacokinetic model of step b) and the pharmacodynamic model of step e) the body of the user (patient) can be divided into a plurality of compartments between which an exchange of the anesthetic substance (drug) occurs. For example, the user (patient) can be divided into an adipose compartment that corresponds to their adipose tissue, a brain compartment that corresponds to the user's (patient's) brain, a plasma compartment that corresponds to the user's (patient's) blood cycle, a muscle that corresponds to the user's (patient's) muscles and organs, a fatty compartment that corresponds to the user's (patient's) fat and connective tissue. Depending on the type of model and the type of medical procedure the user (patient) is undergoing, other compartments may be considered.

Particularly, an anesthetic drug such as Propofol, Fentanyl, Remifentanil, and/or a muscle relaxant drug can be injected intravenously, e.g., by anesthetic fluid infusion pump (6), into the user's (patient's) plasma compartment and, therefore, after injection enters the user's (patient's) bloodstream. From the bloodstream, the drug is distributed within the user's (patient's) body and enters others of their compartments. Within the model, the transfer of drug from one compartment to another can be described, e.g., by so-called transfer rate constants, which indicate the transfer rate between compartments.

The pharmacokinetic and pharmacodynamic profiles of short-acting intravenous agents, such as propofol, remifentanil, alfentanil, or sufentanil, allow for rapid titration of the anesthetic fluid dose until the desired effect is achieved for each user (patient). For example, the course of drug concentration in a three-compartment model for Propofol can be described mathematically by:
Three-exponent equation:

$$Cp = {V}_{1} {e}^{- α t} + {V}_{2} {e}^{- β t} + {V}_{3} {e}^{- γ t}$$

Three-volume distribution
Three clarifications or purifications
Five drug-passage constants

The following table defines the values of the constants for the drug Propofol using the data reported by Marsh and Schnider.

| | | | | Schnider | | | |
|---|---|---|---|---|---|---|---|
| | Marsh | | | | | 70 kg, male, | |
| | General | | | General Model (LBM calculated by | | height: | |
| | Model | 70 kg | | weight, age, gender) | | 170 cm | |
| V1 | 0.228 L*kg⁻¹ | 15.9 | L | 4.27 | L | 4.27 | L |
| V2 | 0.463 L*kg⁻¹ | 32.4 | L | 18.9 − 0.391-(age-53) L | | 24 | L |
| V3 | 2.893 L*kg⁻¹ | 202 | L | 238 | L | 238 | L |

| k₁₀(min⁻¹) | 0.119 | 0.119 | 0.443 + 0.0107*(weight-77) − | 0.384 |
|---|---|---|---|---|
| | | | 0.0159*(LBM-59) + | |
| | | | 0.0062*(height-177) | |
| k₁₂(min⁻¹) | 0.112 | 0.112 | 0.302 − 0.0053*(age-53) | 0.375 |
| k₁₃(min⁻¹) | 0.042 | 0.042 | 0.196 | 0.196 |
| k₂₁(min⁻¹) | 0.055 | 0.055 | [1.29 − 0.024*(age-53)]/[18.9 − | 0.067 |
| | | | 0.391*(age-53)] | |
| k₃₁(min⁻¹) | 0.0033 | 0.0033 | 0.0035 | 0.004 |
| kₑₒ(min⁻¹) | 0.26 | 0.26 | 0.456 | 0.456 |

For example, the pharmacokinetic and pharmacodynamic profiles of short-acting intravenous agents, such as propofol, remifentanil, alfentanil, or sufentanil, allow for rapid titration of the anesthetic fluid dose until the desired effect is achieved for each user (patient). Examples of state models for Propofol are described in Schnider (Schnider et al, 1999, Anesthesiology 90:1502-1516). Similarly, the document (Minto et al, 1997, Anesthesiology 86:24-33) describes models for remifentanil.

Within a pharmacokinetic or pharmacodynamic model, e.g., transfer rate constants indicate the transfer rate between the plasma compartment and the lung compartment, the brain compartment, and other compartments. The time delays of the concentrations in the different compartments are defined through these transfer rate constants. In this case, the plasma compartment represents a linkage mechanism between the different compartments, since drug exchange occurs mainly between the plasma and pulmonary compartments and between the plasma and brain compartments, e.g., but not directly between the pulmonary and cerebral compartment. The volumes of the compartments are also taken into account in the model.

Examples of pharmacokinetic and pharmacodynamic models are described in WO 2005/084731 A2, the content of which is incorporated by reference herein, and in M. Coppens et al. in “Study of the time course of the clinical effect of propofol compared with the time course of the predicted effect-side concentration: performance of three pharmacokinetic-dynamic models.” British Journal of Anesthesia. 104 (4): 452-8 (2010) and by J.-O. Hahn et al. in “A direct dynamic dose-response model of propofol for individualized anesthesia care.” IEEE Transactions on Biomedical Engineering. vol. 59. no. 2. February 2012.

Optionally, some of the embodiments of the method described herein may include a pharmacodynamic processing method based on at least one pre-trained machine learning process selected from the group that includes linear, exponential, logarithmic, logistic, vector support machines, neural networks, KNN, and combinations thereof. One of the technical advantages of these embodiments is that, based on the pharmacokinetic database, the automatic learning process can be trained and retrained, thus achieving better results than deterministic models and models based on static databases of a population.

On the other hand, in step c) the first value (14) of the configuration data (4) contains one or more values of variables of the anesthetic fluid infusion pump (6) that modify the flow of anesthetic fluid administered to the user (patient) to keep them in a state of sedation. This first value (14) is a seed value determined with the pharmacokinetic processing method. However, the pharmacokinetic processing method can consider a pharmacokinetic model that allows for automatically defining the flow of anesthetic fluid that is necessary to maintain the user (patient) in an anesthetic state, so if the model fails or does not adjust correctly to reality, there is a risk that the user feels pain, wakes up or, otherwise, suffers an overdose.

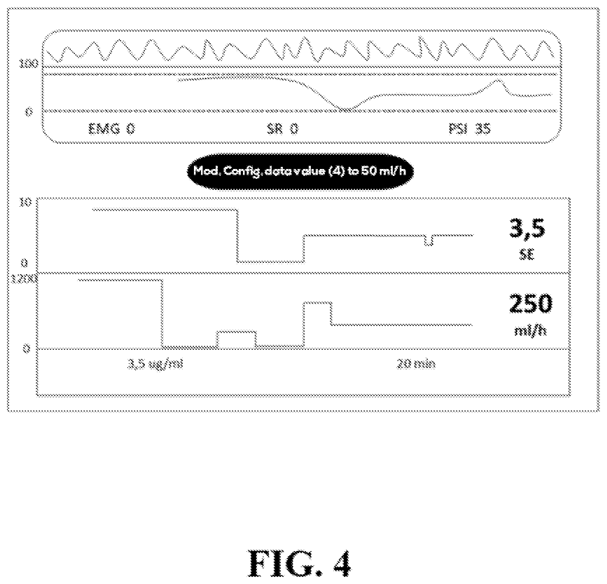

To avoid these problems, the computing unit (1) performs steps c) to g) iteratively, in order to continuously monitor the user (patient) and suggest to the operator (physician) a change in configuration data (4) value (11, 14) that directly affects the flow of anesthetic fluid administered by the anesthetic fluid infusion pump (6). Alternatively, the anesthetic fluid infusion pump (6) can automatically receive the new value (11, 14) of the configuration data (4). However, in order to comply with the law and ethics to which the operator (physician) is subject, it is advisable to have their express authorization before modifying configuration data (4) value (11, 14) in the anesthetic fluid infusion pump (6). For example, FIG. 4 shows an example of a screen in which configuration data (4) value (11, 14) that is suggested to the operator to modify the flow conditions of the anesthetic fluid infusion pump (6) is displayed on a central button.

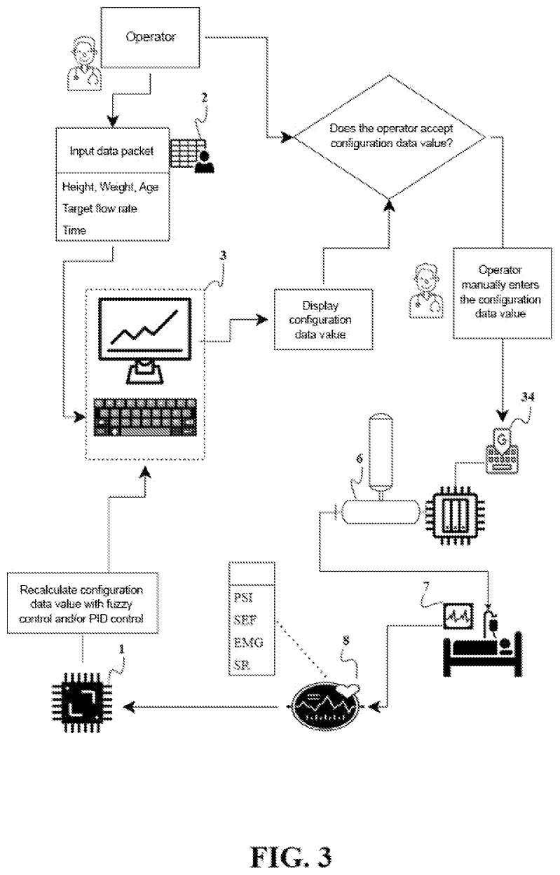

As set forth above, and referring to FIG. 3, in any of the embodiments of the method disclosed herein step c) may include a substep c1) of displaying in the input/output device (3) a notification of the configuration data (4) values (11, 14) to be entered into the anesthetic fluid infusion pump (6), and a substep c2) of receiving in the input/output device (3) an authorization command by the operator, if the operator accepts the configuration data (4) value displayed on the input/output device (3).

Additionally, referring to FIG. 3, in any of the embodiments of the method disclosed herein step c) can also include a substep c3) of receiving from the anesthetic fluid infusion pump (6) a notification that is generated when the operator manually enters the value (11, 14) in said anesthetic fluid infusion pump (6), e.g., through an input/output device (34) of said anesthetic fluid infusion pump (6). As set forth above, in these embodiments of the method, the operator (physician or health professional) can directly enter the value (11, 14) in the anesthetic fluid infusion pump (6), e.g., when it is desired to maintain said anesthetic fluid infusion pump (6) without major amendments, or in order to keep within the operator's functions the responsibility of directly setting the anesthetic fluid flow conditions.

Alternatively, referring to FIG. 1 and FIG. 2, the anesthetic fluid infusion pump (6) can be directly connected to the computing unit (1), so the computing unit (1) sends the configuration data (4) with value (11, 14) to the anesthetic fluid infusion pump (6) at the end of substep c1).

On the other hand, the anesthetic fluid infusion pump (6) is an electromechanical device that controls the flow of medical fluids. Examples of medicine infusion pumps are the AS40A® infusion pump from Baxter Healthcare Inc, and the Atom Syringe pump S-123. This pump drives a medical syringe to deliver a medicine to the patient at a precisely controlled rate. For example, the anesthetic fluid infusion pump (6) can be an active fluid pumping mechanism, i.e., a positive displacement of the syringe plunger, which allows for the fluid to be expelled from the syringe. This type of pump is generally known as a syringe pump. Other examples of the anesthetic fluid infusion pump (6) are positive displacement pumps, linear peristaltic pumps, rotary peristaltic pumps, and other infusion pumps known to a person ordinarily skilled in the art.

On the other hand, the method in step e) generates activation data (9) that is produced when the instrumentation system (7) obtains signals and variables, or derived parameters (e.g., extracted from the signals by statistical treatment, such as the mean, maximum value, minimum value, RMS value, etc.) whose values cause the control process to be executed (12) when compared against a reference threshold range (13).

Referring to FIG. 3, the instrumentation system (7) may include medical devices and sensors configured to monitor the patient's vital signs. For example, the instrumentation system (7) may include an electroencephalography (EEG) monitor that receives signals from electrodes arranged on the user's head. Likewise, the instrumentation system (7) may include a medical monitor, wearables, oximeters, heart rate monitors, blood pressure sensors, and any other device or sensor known to a person ordinarily skilled in the art.

On the other hand, in step f), the control process (12) includes a PID control method and a fuzzy control method that allow for obtaining the new value (e.g., the second value (11)) of the configuration data (4). The foregoing allows for monitoring the state of the user (patient) under anesthesia in real time and obtain new values of the configuration data (4) that modify the anesthetic fluid flow conditions. On the other hand, the predetermined threshold range (13) can be obtained through a pharmacodynamic database.

Step f) can have a substep f1) of obtaining output data by performing a PID (Proportional-Integral-Derivative) control method that takes as input the first value (14) of the configuration data (4), the state (8) and the predetermined threshold (13), and a substep f2) of obtaining the second value (11) of the configuration data (4) performing a fuzzy control method taking as input the output data of the PID control method.

In some of the embodiments of the method disclosed herein, in substep f1), the PID control method obtains the second value (11) of the configuration data (4) as output data, if the difference between the first value (14) and the default threshold (13) is less than a reference value, and it goes to step g), otherwise, the PID control method is canceled and step f2) is executed, where the fuzzy control method obtains the second value (11) of the configuration data (4) taking as input the first value (14) of the configuration data (4), the state data (8) and the predetermined threshold (13). In this way, the PID control method is performed when the difference between the first value (14) and the predetermined threshold (13) is less than a reference value, which is related to the fact that in these situations the non-linearity effects of the pharmacodynamic and/or pharmacokinetic models taken into account by the data processing method (5) and the validation process (10) are not emphasized.

The reference value depends on the type of medicines and anesthetic substance that are pumped in the anesthetic fluid infusion pump (6), and on their pharmacokinetic and pharmacodynamic behavior in the user's (patient's) body.

Additionally, traditional TCI infusion pumps are based on statistical population models and closed-loop systems use mathematical control models such as PID, which is a linear and static method. However, the human body is a non-linear system, with variations over time and, therefore, fuzzy control is an alternative to take into account the non-linearities of human physiology for a safer and more precise control. In this way, by means of the PID control method in combination with the Fuzzy control method, consistency in control actions, minimization of the error in steady state, and acceleration of the dynamic response typical of PID control are obtained, while the non-linearities and variations in time, characteristics of human physiology, are taken into account by the Fuzzy control.

Therefore, when there is a difference between the first value (14) and the predetermined threshold (13) greater than the reference value, the PID control method begins to fail due to the non-linearity of said pharmacodynamic models and/or pharmacokinetics taken into account by the data processing method (5) and the validation process (10). In this case, the fuzzy control model allows for a better adjustment to reality and allows for obtaining a second value (11) of the configuration data (4) that is more reliable than the one obtained by the PID control method.

This also has the technical effect that performing the PID control method has a lower computational cost, in terms of processing power and memory consumption, than the Fuzzy control method. Therefore, there is a more efficient method and system by selectively performing said PID and Fuzzy control methods.

On the other hand, the present invention also describes a system for controlling a flow of anesthetic fluid, comprising a computing unit (1) configured to perform any of the embodiments of the method disclosed herein.

For example, the system disclosed herein can be understood as a clinical decision support system (CDSS) for real-time control of total intravenous anesthesia (TIVA), based on a fuzzy logic pharmacodynamic model.

The computing unit (1) can be selected from the group that includes microcontrollers (e.g., arduino, Raspberri-pi), microprocessors, DSCs (Digital Signal Controller), FPGAs (Field Programmable Gate Array), CPLDs (Complex Programmable Logic Device), ASICs (Application Specific Integrated Circuit), SoCs (System on Chip), PSoCs (Programmable System on Chip), computers, servers, tablets, cell phones, smart phones, signal generators, and other similar or equivalent computing units known by a person ordinarily skilled in the art.

The computing unit (1) may include a memory module configured to store the input data packet (2), and the historical record of the configuration data (4) values (11, 14). The memory module can be selected from the group that includes, RAM memories (cache, SRAM, DRAM, DDR), ROM memory (Flash, Cache, Hard Drives, SSD, EPROM, EEPROM, removable ROM memories (e.g., SD (miniSD, microSD, etc), MMC (MultiMedia Card), Compact Flash, SMC (Smart Media Card), SDC (Secure Digital Card), MS (Memory Stick), among others)), CD-ROM, Digital Versatile Discs (DVDs) or other optical storage, magnetic cassettes, magnetic tapes, storage or any other media that can be used to store information to be accessed by the computing unit (1) that are known to a person ordinarily skilled in the art.

In addition, the system may include the input/output device (3) and a communications module configured to exchange data with the anesthetic fluid infusion pump (6) and/or with the instrumentation system (7). The communication module is selected from the group consisting of wired communication modules, wireless communication modules, and wired and wireless communication modules.

Examples of wireless communication modules are modules that use a wireless communication technology that is selected from the group consisting of Bluetooth, WiFi, Radio Frequency RF ID (standing for Radio Frequency Identification), UWB (Ultra-Wide Band), GPRS, Konnex or KNX, DMX (Digital MultipleX), WiMax and equivalent wireless communication technologies that are known to a person ordinarily skilled in the art and combinations thereof.

Similarly, a wired communications module has a wired connection port that allows for communication with external devices through a communications bus, which is selected, among others, from the group made up of I2C (for the acronym of IIC Inter-Integrated Circuit), CAN (Controller Area Network), Ethernet, SPI (Serial Peripheral Interface), SCI (Serial Communication Interface), QSPI (Quad Serial Peripheral Interface), 1-Wire, D2B (Domestic Digital Bus), Profibus and others known to a person ordinarily skilled in the art. For example, this type of communication module can be used to connect the computing unit (1) with the anesthetic fluid infusion pump (6).

Additionally, the computing unit (1) can communicate with the anesthetic fluid infusion pump (6) and/or with the instrumentation system (7) through one or more communication protocols selected between AS-i in accordance with the standard International IEC62026-2, Bristol Standard Asynchronouss Protocol (BSAP), CC-Link Industrial Networks, CIP (Common Industrial Protocol), CAN bus (Controlled Area Network) such as CANopen and DeviceNet, ControlNet, DF-1, DirectNET, EtherCAT, Ethernet Global Data (EGD), Ethernet Powerlink, EtherNet/IP, FINS FOUNDATION type fieldbus (e.g., H1, HSE), GE SRTP (Service Request Transport Protocol), HART (Highway Addressable Remote Transducer) protocol, Intelligent Distributed System (Honeywell SDS), HostLink, INTERBUS, IO-Link, MECHATROLINK, MelsecNet, Modbus, Modbus RTU, Modbus ASCII, Modbus TCP/IP or Modbus TCP, Modbus over TCP/IP or Modbus over TCP or Modbus RTU/IP, Modbus over UDP, Modbus Plus (Modbus+, MB+ or MBP), Pemex Modbus, Enron Modbus, Optomux, Process Image Exchange Protocol (PieP), Profibus, PROFINET IO, RAPIEnet (Real-time Automation Protocols for Industrial Ethernet), SERCOS interface, SERCOS III, Sinec H1, SynqNet, or Time-Triggered Ethernet (SAE AS6802).

On the other hand, the input/output device (3) may include a storage device, display device and/or a Human Interface Device (HID).

Examples of display device include, without limitation, any device that can be connected to a computing unit and display its output selected from, but not limited to, a CRT (Cathode Ray Tube) monitor, flat panel display, LCD Liquid Crystal Display, Active Matrix LCD display, Passive Matrix LCD display, LED Displays, Screen Projectors, TV (4KTV, HDTV, Plasma TV, Smart TV), OLED (Organic Light Emitting Diode) displays, AMOLED (Active Matrix Organic Light Emitting Diode) displays, Quantum Dot displays QD (Quantic Display), segment displays, among other devices capable of displaying data to a user, known to those skilled in the art, and combinations thereof.

Examples of the HID device (Human Interface Device) can be without limitation, keyboard, mouse, trackball, touchpad, pointing device, joystick, touch screen, among other devices that are capable of allowing a user to enter data on the computation unit of the device and combinations thereof.

On the other hand, the present invention also relates to a computer program comprising instructions, which when the program is executed in a system pursuant to any of the embodiments previously described, cause said system to perform the steps of a method according to any of the embodiments of the methods previously described in this invention.

The computer program may be written in any programming language or framework known by a person ordinarily skilled in the art. For example, the computer program may be written in Java, JavaScript, Perl, PHP, and C++, #C, Python, R-studio SQL, Swift, Ruby, Delphi, Visual Basic, D, HTML, HTML5, CSS, NodeJs, Angular, React, Go, RPA, .NET, Scala, Drupal, Ember, Labview and other programming languages and/or frameworks known by a person ordinarily skilled in the art.

On the other hand, the present invention also relates to a computer program comprising instructions, which cause said system to perform the steps of a method according to any of the embodiments of the methods previously described in this invention, when the program is executed in a system according to any of the embodiments previously described.

The memory module can be selected from the group that includes, RAM memories (cache, SRAM, DRAM, DDR), ROM memory (Flash, Cache, Hard Drives, SSD, EPROM, EEPROM, removable ROM memories (e.g., SD (miniSD, microSD, etc.), MMC (MultiMedia Card), Compact Flash, SMC (Smart Media Card), SDC (Secure Digital Card), MS (Memory Stick), among others)), CD-ROM, Digital Versatile Discs (DVDs) or other optical storage, magnetic cassettes, magnetic tapes, storage or any other media that can be used to store information and can be granted access by a processing unit.

As set forth above, the methods and systems disclosed herein make it possible to eliminate the use of anesthetic gases due to their proven adverse effects for the environment and for patients. In the same way, the control based on the state of the user (patient) under anesthesia taking into account variables (contained in the state data (8)) that reflect a more complete state of brain function compared to only monitoring data from EEG comparing its static value and scale. On the other hand, the methods and systems disclosed herein make it possible to take into account the trend that these values have had in each user (patient) and in each procedure. For example, with these trends and values, the pharmacodynamic model applied in the computer-implemented method described herein can interpret and calculate, by means of fuzzy logic, the new target configuration data (4) value and the new infusion rate that will be suggested to the operator (physician).

### EXAMPLES

### Example 1

In a first example of the method described herein, in step a) the computing unit (1) receives an input data packet (2) with data on height, weight, age, initial target concentration of propofol (anesthetic substance of this example), peak waiting time to measure the induced effect, and expected duration of a medical procedure undergone by the user (patient). These data are entered by an operator (healthcare professional).

Then, the method goes to step b) in which a first value (14) of the configuration data (4) is obtained. In this example, the first value (14) includes a flow rate value of the anesthetic fluid infusion pump (6). The data processing method (5) includes a pharmacokinetic processing method that takes into account a pharmacokinetic model based on a population of users.

In this example, the anesthetic fluid infusion pump (6) is a positive displacement syringe pump, such as the AS40A® from Baxter Healthcare Inc.

Each time step c) is executed, the operator enters the configuration data (4) into the anesthetic fluid infusion pump (6). In step d), the state data (8) is obtained from an instrumentation system (7) that includes a monitor configured to obtain EEG, PSI, SEF, SR, and EMG variables.

Based on the state data (8), step e) the computing unit (1) compares the values of EEG, PSI, SEF, SR, and EMG taking into account the predetermined threshold range (13) related to an anesthetic state of the user. The predetermined threshold (13) is obtained by performing a pharmacodynamic processing method that takes as input the input data packet (2) and a pharmacodynamic database. The default threshold (13) includes acceptable ranges of EEG, PSI, SEF, SR, and EMG values consistent with an anesthetic state or also called therapeutic window (RTEB-Real Time Effect on Brain) of the user.

In step f), the computing unit (1) obtains the new value (11, 14) of the configuration data (4) from the control process (12), applying the PID control method and the fuzzy control method according to the variation of the values of EEG, PSI, SEF, SR, and EMG with respect to the predetermined threshold (13).

### Example 2

A system to control a flow of anesthetic fluid was designed and built, which includes a computing unit (1) that for this example is a specific-purpose computer that includes an input/output device (3) having a screen and a keyboard. The computing unit (1) is configured to perform the method of example 1.

### Example 3

The method and the system of examples 1 and 2 were modified, so that the computing unit (1) is connected by means of a wired communications protocol. The computing unit (1) has a communications module that bidirectionally converts RS-232 serial signals into USB.

It shall be understood that the present invention is not limited to the embodiments described and illustrated because, as will be evident to a person versed in the art, there are possible variations and modifications that do not depart from the spirit of the invention, which is only defined by the following claims.

1. A computer-implemented method for controlling a flow of anesthetic fluid that comprises executing in a computing unit (1) the following steps:
a) Receiving an input data packet (2) including demographic data and physiological data of a user, and target concentration data of an anesthetic substance,
wherein the input data packet (2) is entered by an operator through an input/output device (3) connected to the computing unit (1):
b) Obtaining a first value (14) of a configuration data (4) by performing a data processing method (5) configured to perform a pharmacokinetic processing method that takes as input the input data packet (2) and a database pharmacokinetic data;
c) Inputting the first configuration data (4) into an anesthetic fluid infusion pump (6), wherein the first configuration data (4) sets anesthetic fluid flow operating conditions:
d) Receiving state data (8) from an instrumentation system (7)
wherein the state data (8) includes records of at least one variable selected from the group that includes state variables of the user under anesthesia, electroencephalographic signals, electromyography signals, data derived from electroencephalographic and/or electromyography signals, and combinations thereof:
e) Obtaining an activation data (9) by means of a validation process (10) that takes the state data (8) as input and a predetermined threshold range (13) related to an anesthetic state of the user,
wherein the activation data (9) is generated when at least one of the state data variables (8) has a value outside the predetermined threshold range (13),
wherein the predetermined threshold range (13) is obtained from a pharmacodynamic database:
f) Obtaining a second value (11) of the configuration data (4) performing a control process (12) that includes the substeps:
f1) Obtaining an output data by performing a PID (Proportional-Integral-Derivative) control method that takes as input the first value (14) of the configuration data (4), the state data (8) and the default threshold (13), and
f2) Obtaining the second value (11) of the configuration data (4) performing a fuzzy control method taking as input the output data of the PID control method, and
g) Sending the configuration data (4) with a second value (11) to the anesthetic fluid infusion pump (6) to modify the anesthetic fluid flow conditions; and
h) Repeating steps c) to g).

2. The method of claim 1, which further comprises in step c) the following substeps:
c1) Displaying in the input/output device (3) a notification of the configuration data (4) values (11, 14) to be entered in the anesthetic fluid infusion pump (6), and
c2) Receiving in the input/output device (3) an authorization command from the operator, if the operator accepts the configuration data (4) value displayed in the input/output device (3).

3. The method of claim 2, which further comprises a substep c3) of receiving from the anesthetic fluid infusion pump (6) a notification that is generated when the operator manually enters the value (11, 14) in said anesthetic fluid infusion pump (6).

4. The method of claim 2, wherein the computing unit (1) sends the configuration data (4) with value (11, 14) to the anesthetic fluid infusion pump (6) at the end of substep c1).

5. The method of claim 1, wherein the input data packet (2) includes at least one data selected from the group comprising weight, sex, age, height, medical history data, and combinations thereof.

6. The method of claim 1, wherein the target concentration data is a concentration value of an anesthetic substance selected from the group including hypnotic, analgesic, and amnestic intravenous anesthetic drugs, paralytic agents, vasodepressors, pressor substances, and formulations with combinations thereof.

7. The method of claim 1, wherein the data processing method (5) of step b) includes a pharmacokinetic processing method based on a pharmacokinetic model that correlates the elimination of the anesthetic substance in the body of a user.

8. The method of claim 1, wherein in substep f1) the PID control method obtains the second value (11) of the configuration data (4) as output data, if the difference between the first value (14) and the predetermined threshold (13) is less than a reference value, and go to step g), otherwise,
the PID control method is canceled and step f2) is executed, wherein the fuzzy control method obtains the second value (11) of the configuration data (4) taking the first value (14) of the configuration data (4), the state data (8), and the default threshold (13).

9. A system for controlling a flow of anesthetic fluid, comprising:
a computing unit (1) configured to perform the method of claim 1.

10. A computer-readable media that includes instructions that, when executed by a computing unit (1), causes said computing unit (1) to perform the method of claim 1.
